(19) United States
(12) Patent Application Publication
(10) Pub. No.: US 2013/0237559 A1
(43) Pub. Date: Sep. 12, 2013

(54) SUSTAINED RELEASE COMPOSITION COMPRISING AN AMINE AS ACTIVE AGENT AND A SALT OF A CYCLIC ORGANIC ACID

(71) Applicants: Ronnie Ortiz, Apple Valley, MN (US); James S. Jensen, Edina, MN (US); Kristopher R. Lundell, New Hope, MN (US); Victoria Ann O'Neill, Wayzata, MN (US); Sean Mahoney, Plymouth, MN (US)

(72) Inventors: Ronnie Ortiz, Apple Valley, MN (US); James S. Jensen, Edina, MN (US); Kristopher R. Lundell, New Hope, MN (US); Victoria Ann O'Neill, Wayzata, MN (US); Sean Mahoney, Plymouth, MN (US)

(21) Appl. No.: 13/807,174

(22) PCT Filed: Jun. 29, 2011
(86) PCT No.: PCT/US11/42428

§ 371 Date: Apr. 15, 2013

Related U.S. Application Data
(60) Provisional application No. 61/360,179, filed on Jun. 30, 2010.

(51) Int. Cl. A61K 31/485; A61K 31/192; A61K 31/135

(52) U.S. Cl. CPC A61K 31/485; A61K 31/135; A61K 31/192
USPC 514/289; 514/646; 514/570

(57) ABSTRACT

The present invention provides sustained-release oral pharmaceutical compositions and methods of use. The sustained-release oral pharmaceutical compositions include an amine-containing compound (e.g., an opioid) (including salts thereof) and a pharmaceutically acceptable salt of a non-NSAID cyclic organic acid compound.

### RELATED APPLICATION DATA

This application claims the benefit of U.S. Provisional Application Ser. No. 61/360,179, filed Jun. 30, 2010, and titled ORAL PHARMACEUTICAL COMPOSITIONS COMPRISING AN AMINE-CONTAINING COMPOUND AND A SALT OF A CYCLIC ORGANIC ACID, which is hereby incorporated by reference in its entirety.

### BACKGROUND

For many pharmacologically active compounds, immediate-release formulations are characterized by a short duration of action, typically necessitating frequent administrations in order to maintain therapeutic levels of the compounds in patients. Thus, there is a need for new oral pharmaceutical compositions that provide sustained release, and ideally zero-order release kinetics, and less frequent dosing.

While complex dosage forms such as mechanical pumps, osmotic pumps, implantable devices, and the like, have been purported to achieve near zero-order release kinetics, the methods for achieving a constant sustained release using those dosage forms are costly to scale up and manufacture, thereby limiting their commercial viability.

### SUMMARY

The present invention provides sustained-release oral pharmaceutical compositions and methods of use. These compositions can be readily scaled up and manufactured using existing traditional and cost-effective technologies.

In one embodiment, the present invention provides a sustained-release oral pharmaceutical composition comprising within a single dosage form: a hydrophilic matrix; a pharmacologically active amine-containing compound (in certain embodiments, this is an opioid (including salts thereof), and in certain embodiments, this is a non-opioid amine-containing compound (including salts thereof)); and a pharmaceutically acceptable salt of a non-NSAID cyclic organic acid compound (i.e., “salt of a cyclic organic acid”); wherein the amine-containing compound (including salts thereof) and the salt of the cyclic organic acid are within the hydrophilic matrix; wherein the composition exhibits a release profile comprising a substantial portion that is representative of zero-order release kinetics (with respect to the amine-containing compound) under in vitro conditions.

In another embodiment, the present invention provides a sustained-release oral pharmaceutical composition comprising within a single dosage form: a hydrophilic matrix; a pharmacologically active amine-containing compound (in certain embodiments, this is an opioid (including salts thereof), and in certain embodiments, this is a non-opioid amine-containing compound (including salts thereof)); a pharmaceutically acceptable salt of a non-NSAID cyclic organic acid compound; and a pharmaceutically acceptable anionic surfactant; wherein the amine-containing compound (including salts thereof), the salt of the cyclic organic acid, and the anionic surfactant are within the hydrophilic matrix. Preferred such compositions exhibit a release profile comprising a substantial portion that is representative of zero-order release kinetics under in vitro conditions.

The present invention also provides methods of providing a desired effect by administering to a subject a composition of the present invention. In methods of the present invention, administering a composition of the present invention comprises administering once or twice per day, and often once per day.

Herein, an “NSAID” is a salt of a non-steroidal anti-inflammatory drug. These are drugs with analgesic, antipyretic and, in higher doses, anti-inflammatory effects. NSAIDs are sometimes also referred to as non-steroidal anti-inflammatory agents/analgesics (NSAIAs) or non-steroidal anti-inflammatory medicines (NSAIMs). As used herein, the term “NSAID” refers only to nonspecific COX inhibitors. There are roughly seven major classes of NSAIDs, including: (1) salicylate derivatives, such as acetylsalicylic acid (aspirin), amoxiprin, benorylate/benorilate, choline magnesium salicylate, diflunisal, ethenzamide, faislamine, methyl salicylate, magnesium salicylate, salicyl salicylate, and salicylamide; (2) 2-aryl propionic acid derivatives, such as ibuprofen, ketoprofen, alminoprofen, carprofen, dexibuprofen, dexketoprofen, fenbufen, fenoprofen, flunoxaprofen, flurbiprofen, ibuproxam, ondoprofen, ketorolac, loxoprofen, naproxen, oxaprozin, pirprofen, suprofen, and tiaprofenic acid; (3) pyrazolidine derivatives, such as phenylbutazone, ampyrone, azapropazone, clofezone, kebuzone, metamizole, mofebutazone, oxyphenbutazone, phenazone, and sulfinpyrazone; (4) N-arylanthranilic acid (or fenamate) derivatives, such as mefenamic acid, flufenamic acid, meclofenamic acid, tolfenamic acid, and esters thereof; (5) oxicam derivatives, such as piroxicam, droxicam, lornoxicam, meloxicam, and tenoxicam; (6) arylalkanoic acids, such as diclofenac, aceclofenac, acemethacin, alclofenac, bromfenac, etodolac, indomethacin, nabumetone, oxametacin, proglumetacin, sulindac (prodrug), and tolmetin; (7) indole derivatives, such as indomethacin.

Herein, a “non-NSAID” is a compound that is not classified as an NSAID. Although acetaminophen (paracetamol) is an analgesic and it is sometimes grouped with NSAIDs, it is not an NSAID because it does not have any significant anti-inflammatory activity. However, it is not a cyclic organic acid as defined herein because it is an extremely weak acid (pKa 9.7) and is not easily ionizable; thus, it is not suitable for the purposes of the present invention.

Herein, a “hydrophilic matrix” refers to a “gel forming” or “hydrogel” material wherein upon administration the hydrophilic matrix slowly expands to form a gel upon exposure to liquids. Likewise, the hydrophilic matrix swells and forms a gel upon exposure to an aqueous environment, such as, e.g., in an in vitro dissolution test.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a composition comprising “a” salt of a non-steroidal anti-inflammatory drug can be interpreted to mean that the composition includes “one or more” non-steroidal anti-inflammatory drugs. Similarly, a composition comprising “a” pharmaceutically acceptable anionic surfactant can be interpreted to mean that the composition includes “one or more” pharmaceutically acceptable anionic surfactants.

As used herein, the term “or” is generally employed in its usual sense including “and/or” unless the content clearly dictates otherwise. The term “and/or” means one or all of the listed elements or a combination of any two or more of the listed elements.

Also herein, all numbers are assumed to be modified by the term “about” and preferably by the term “exactly.” Notwithstanding that the numerical ranges and parameters setting forth the broad scope of the invention are approximations, the numerical values set forth in the specific examples are reported as precisely as possible. All numerical values, however, inherently contain certain errors necessarily resulting from the standard deviation found in their respective testing measurements.

Also herein, the recitations of numerical ranges by endpoints include all numbers subsumed within that range (e.g., 1 to 5 includes 1, 1.5, 2, 2.75, 3, 3.80, 4, 5, etc.). Where a range of values is “up to” a particular value, that value is included within the range.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIGS. 1 through 4 show dissolution profiles in phosphate buffer for certain dextromethorphan (DXM) formulations in accordance with embodiments of the present invention.

FIGS. 5 and 6 show dissolution profiles in phosphate buffer for certain comparative dextromethorphan (DXM) formulations.

FIG. 7 shows a dissolution profile in phosphate buffer for a tramadol (TMD) formulation in accordance with embodiments of the present invention.

FIG. 8 shows a dissolution profile in phosphate buffer for a comparative tramadol (TMD) formulation.

FIG. 9 shows a dissolution profile in phosphate buffer for a certain dextromethorphan (DXM) formulation in accordance with embodiments of the present invention.

### DETAILED DESCRIPTION OF ILLUSTRATIVE EMBODIMENTS

The present invention provides sustained-release oral pharmaceutical compositions and methods of use. Preferably, such compositions are used for pain treatment, cough suppression, muscle relaxation, treatment of migraine headaches, spasms, convulsions, antihistamine effect, or other indications. Such compositions are in a single dosage form and include a pharmacologically active amine-containing compound (including salts thereof), a pharmaceutically acceptable salt of a non-NSAID cyclic organic acid compound, and a hydrophilic matrix. Certain embodiments also include a pharmaceutically acceptable anionic surfactant.

Herein, sustained-release compositions release the amine-containing compound (herein, the term “compound” includes within its scope salts) over a period of time greater than 60 minutes, generally much greater than 60 minutes. Preferred sustained-release formulations demonstrate at least 60%, and more preferably at least 80%, release of the amine-containing compound over a desired period (e.g., a period of 8 to 12 hours). If desired, however, the formulations of the present invention could be tailored to release the amine-containing compound over any period from 6 hours to 24 hours or longer.

First-order release is often observed for sustained-release compositions that have been described in the literature of the field. In particular, first-order release is expected for typical hydrophilic matrix formulations. First-order release results from a mechanism where the instantaneous rate of release is dependent on the quantity or concentration of the compound of interest remaining in the dosage form. The instantaneous rate is therefore greatest in the early part of a dissolution profile, and the instantaneous rate becomes progressively lessened over time.

In contrast, zero-order release is typically observed where the rate of release is independent of the quantity or concentration of the compound of interest remaining in the dosage form. The instantaneous rate of release therefore remains relatively unchanged over time. A true zero-order dissolution profile would accordingly be a straight line from zero percent release (at time=0) to 100 percent release.

Particularly preferred sustained-release compositions of the present invention demonstrate a zero-order release profile with respect to the amine-containing compound under in vitro conditions, such as when tested in accordance with appropriate test methods (e.g., methods provided in United States Pharmacopeia). In particular, the sustained-release compositions of the present invention demonstrate a retarded rate of release in the early stages (i.e., up to at least 50% total release, and preferably up to at least 60% total release) of a dissolution profile, as compared to a similar formulation that does not contain the salt of a cyclic organic acid.

Herein, “zero-order” with respect to the amine-containing compound (including salts thereof) means a relatively constant rate of release (i.e., exhibiting a substantially linear release profile over a period of time, preferably at least a few hours). Although a portion (e.g., the initial 30-60 minutes) of the release profile may not be zero-order a substantial portion (e.g., several hours), and preferably a major portion, of the release profile is representative of zero-order release kinetics. It should be noted that in the practice of the invention, the very late stages of a dissolution profile may not be representative of zero-order release, such as after 80% or 90% total release has been achieved; however, in that event a substantial portion of the release profile would be representative of zero-order release kinetics.

For example, release profiles that have a linear regression r² value of 0.9873, 0.958, and 0.9696 are considered zero-order. Preferably, zero-order refers to a release profile that has a linear regression r² value of at least 0.93. By comparison, release profiles that have a linear regression r² value of 0.9271, 0.9199, or lower (e.g., 0.7017 and 0.8760) show significant deviation from the linear fit model for zero-order release. Other methods of statistical analysis are further able to distinguish first-order release from zero-order release. For example, nonlinear regression methods could be employed. In the practice of the present invention, it would be expected that a method such as nonlinear regression as applied to the dissolution data would result in a model that is much closer to zero-order release than first-order release for a substantial, and preferably major, portion of the release profile.

Furthermore, dosage forms could be purposefully designed to include an immediate-release coating, or a bilayer or multi-layer formulation comprising an immediate-release layer, while practicing the present invention, without a true zero-order release being observed throughout the dissolution profile; nevertheless, a substantial portion of the release profile would be expected to be representative of zero-order release kinetics.

### Amine-Containing Compounds

The amine-containing compounds of the present invention are pharmacologically active compounds (i.e., used to prevent or treat a condition, for example as a dietary supplement) that include one or more amine groups (primary, secondary, tertiary amines, or combinations thereof). In certain preferred embodiments, the amine-containing compound comprises a tertiary amine. In certain embodiments, the amine-containing compound comprises a ring nitrogen that is a tertiary amine. In other preferred embodiments, the amine-containing compound comprises a tertiary amine or a secondary amine, or a combination thereof. In yet other embodiments, the amine-containing compound comprises two or more of a tertiary amine, a secondary amine, and a primary amine. Typically, such amine-containing compounds include opioid and non-opioid compounds. Furthermore, the term “compound” as used herein includes salts thereof.

A pharmacologically active amine-containing compound (e.g., an opioid, particularly an opioid analgesic) is used herein in an amount that provides the desired effect. Preferably, this is a therapeutically effective amount. Determination of an effective amount will be determined by the condition being treated (e.g., pain, cough, spasms, migraine headaches, and the like) and on the target dosing regimen (e.g., once per day, twice per day). Determination of such an amount is well within the capability of those skilled in the art, especially in light of the detailed disclosure provided herein. For example, if the composition is used as a cough suppressant, the amount of the opioid would be that which is effective for suppressing a cough. If the composition is used to treat pain, for example, a therapeutically effective amount of an opioid is referred to herein as a “pain-reducing amount.” Herein, this means an amount of compound effective to reduce or treat (i.e., prevent, alleviate, or ameliorate) pain symptoms over the desired time period. This amount can vary with each specific amine-containing compound depending on the potency of each. For example, for hydrocodone, the amount per single dosage form of the present invention may be 5 mg to 50 mg.

### Amine-Containing Compounds: Opioids

An opioid is a chemical substance that works by binding to opioid receptors, which are found principally in the central nervous system and the gastrointestinal tract. The receptors in these two organ systems mediate both the beneficial effects, and the undesirable side effects. There are three principal classes of opioid receptors, μ, κ, δ (mu, kappa, and delta), although up to seventeen have been reported, and include the s, ι, λ, and ξ (Epsilon, Iota, Lambda and Zeta) receptors. There are three subtypes of μ receptor: μ₁ and μ₂, and the newly discovered μ₃. Another receptor of clinical importance is the opioid-receptor-like receptor 1 (ORL1), which is involved in pain responses as well as having a major role in the development of tolerance to μ-opioid agonists used as analgesics. An opioid can have agonist characteristics, antagonist characteristics, or both (e.g., pentazocine is a synthetic mixed agonist-antagonist opioid analgesic of the benzomorphan class of opioids used to treat mild to moderately severe pain). The main use for opioids is for pain relief, although cough suppression is also a common use. For example, hydromorphone is used to relieve moderate to severe pain and severe, painful dry coughing. Hydrocodone is most commonly used as an intermediate-strength analgesic and strong cough suppressant.

There are a number of broad classes of opioids: natural opiates, which are alkaloids contained in the resin of the opium poppy, and include morphine and codeine; semi-synthetic opiates, created from the natural opioids, such as hydromorphone (found in Dilaudid), hydrocodone (found in Vicodin), oxycodone (found in Oxycontin and Percocet), oxymorphone, desomorphine, diacetylmorphine (Heroin), nicomorphine, buprenorphine, dihydrocodeine, and benzylmorphine; and fully synthetic opioids, such as fentanyl, methadone, tramadol, and propoxyphene (found in Darvon and Darvocet N). Other examples of opioids include levorphanol, meperidine (found in Demerol), pentazocine, tilidine, and others disclosed, for example, at www.opioids.com.

Certain opioids have antagonist action. For example, naloxone is a g-opioid receptor competitive antagonist. Naloxone is a drug used to counter the effects of opioid overdose, for example heroin or morphine overdose. Naltrexone is an opioid receptor antagonist used primarily in the management of alcohol dependence and opioid dependence. N-methyl naltrexone is also an opioid receptor antagonist.

Various combinations of such compounds can be used if desired. Each of these compounds includes a tertiary amine as shown, wherein the amine nitrogen may or may not be within a ring:

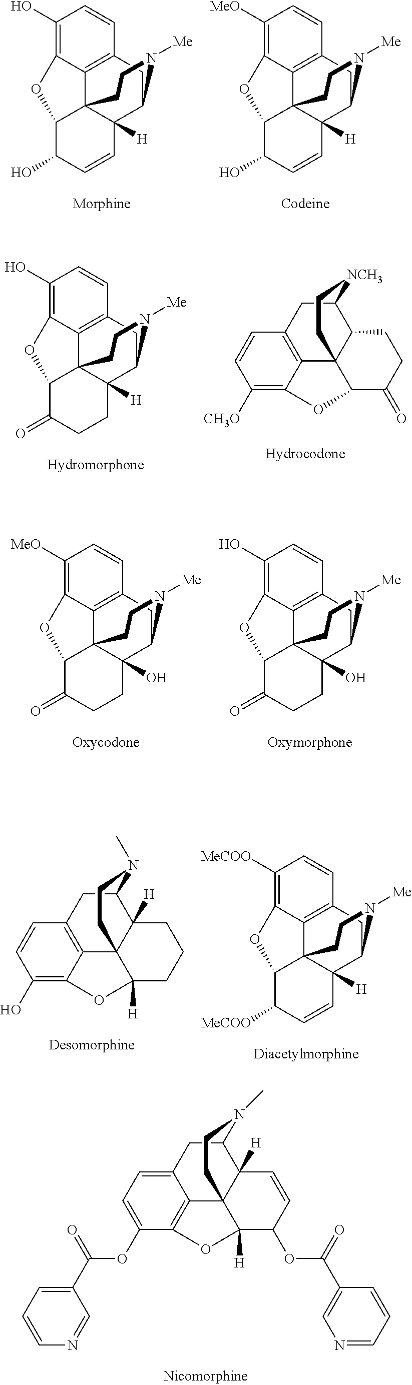

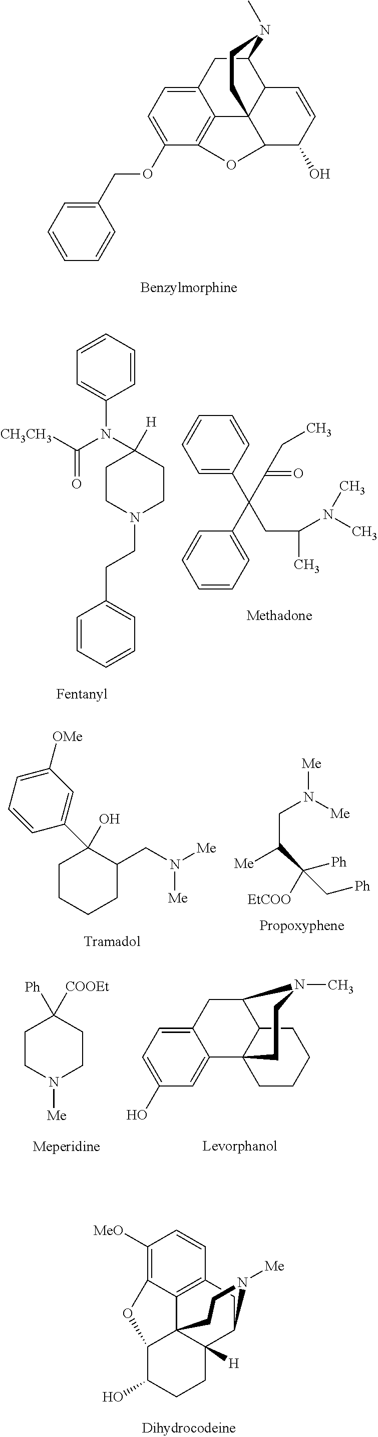

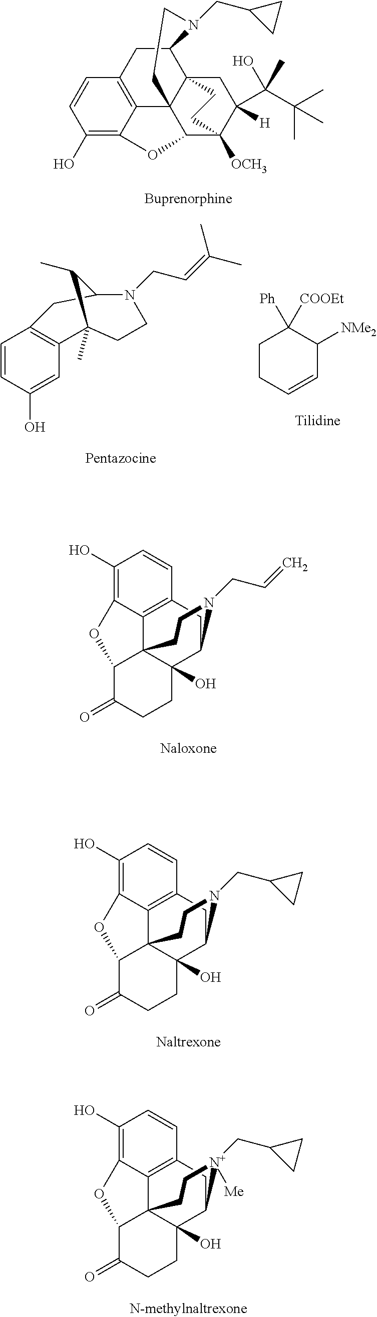

Preferred opioids are opioid analgesics, which have morphine-like activity and produce bodily effects including pain relief and sedation. For certain embodiments, the opioid, particularly opioid analgesic, selected for use in compositions of the present invention is one having a tertiary amine nitrogen. For certain embodiments, the opioid, particularly opioid analgesic, selected includes a ring nitrogen that is a tertiary amine.

The opioids can be used in a variety of salt forms including “pharmaceutically acceptable salts.” Preparation of such salts is well-known to those skilled in pharmaceuticals. Examples of suitable pharmaceutically acceptable salts include, but are not limited to, hydrochlorides, bitartrates, acetates, palmitates, stearates, oleates, hydrobromides, sulfates, tartrates, citrates, maleates, and the like, or combinations of any of the foregoing. Preferably, the opioid is selected from the group consisting of hydrocodone (e.g., hydrocodone bitartrate), tramadol (e.g., tramadol hydrochloride), and combinations thereof. For certain embodiments, the opioid is hydrocodone (particularly hydrocodone bitartrate). For certain embodiments, the opioid is tramadol (particularly tramadol hydrochloride).

### Amine-Containing Compounds: Non-Opioids

Non-opioid compounds are compounds do not bind to opioid receptors in the same way or at the same level as that of opioids. That is, although compounds used in the present invention include one or more amine groups (which may be a primary, secondary, or tertiary amine), and certain compounds used in the present invention include a tertiary amine nitrogen, which may include a ring nitrogen, such compounds used herein are not typically characterized as opioids as they do not have any significant amount of opioid activity.

Various non-opioid amine-containing compounds can be used in the practice of the invention. Each of these compounds includes a tertiary amine as shown, wherein the amine nitrogen may or may not be within a ring:

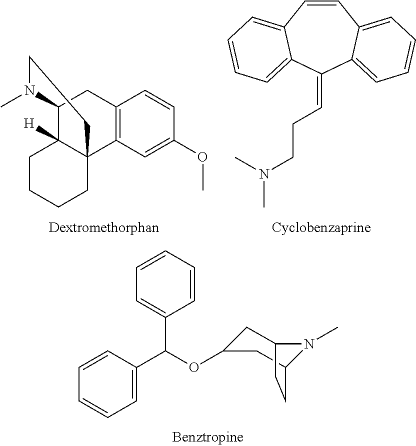

Dextromethorphan (DXM or DM, (+)-3-methoxy-17-methyl-9α,13α,14α-morphinan) is an antitussive drug used primarily as a cough suppressant, for the temporary relief of cough caused by minor throat and bronchial irritation (as commonly accompanies the common cold), as well as those resulting from inhaled irritants. Its mechanism of action is as an NMDA receptor antagonist.

Cyclobenzaprine (3-(5H-dibenzo[a,d]cyclohepten-5-ylidene)-N,N-dimethyl-1-propanamine) is a muscle relaxant that works in the central nervous system by blocking nerve impulses sent to the brain. It is used to treat skeletal muscle conditions such as pain and muscle spasms. The mechanism of action is unknown, although some research indicates that it inhibits the uptake of norepinephrine and blocks 5-HT2A and 5-HT2C receptors. It is also prescribed as a sleep-aid.

Benztropine ((3-endo)-3-(diphenylmethoxy)-8-methyl-8-azabicyclo[3.2.1]octane) is an anticholinergic drug principally used for the treatment of Parkinson's disease.

Other pharmacologically active amine-containing (non-opioid) compounds that may be useful in the practice of the present invention include the following:

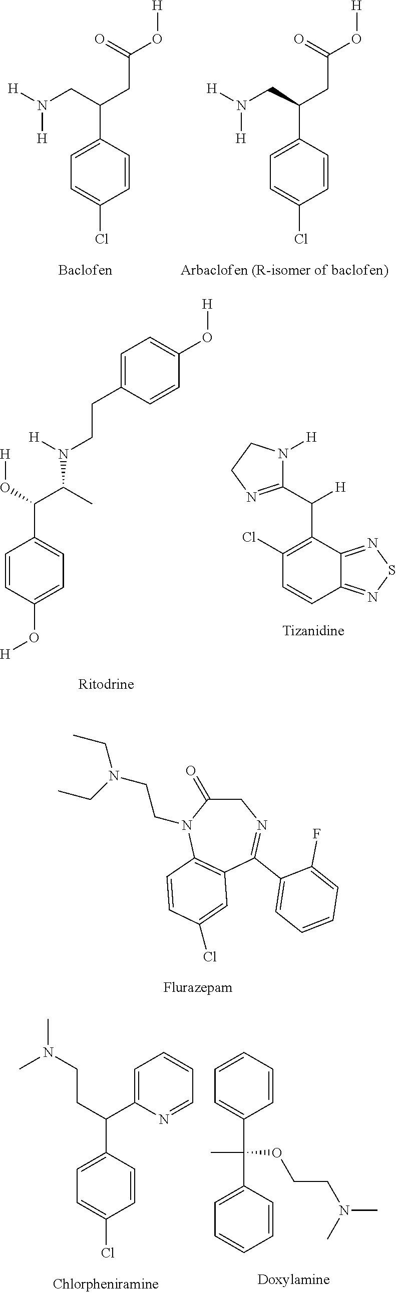

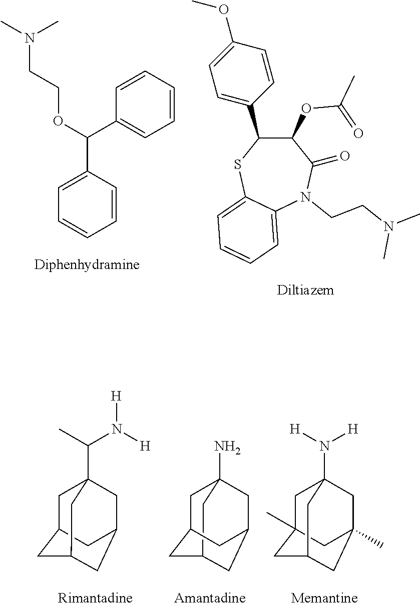

Such compounds function, for example, as muscle relaxants (baclofen, arbaclofen, ritodrine), antispasmodics (tizanidine), anticonvulsants (flurazepam), antihistamines (chlorpheniramine, doxylamine, and diphenhydramine), as treatment and/or prevention agents for migraine headaches (diltiazem), as antihypertensive agents (diltiazem), antivirals (rimantadine, amantadine), and/or as treatment of Parkinson's Disease (rimantadine, amantadine) or Alzheimer's Disease (memantine).

Mixtures or combinations of suitable amine-containing compounds may also be employed in the practice of the invention. That is, more than one pharmacologically active amine-containing compound may be incorporated into one dosage form.

The amine-containing compounds can be used if desired in a variety of salt forms including “pharmaceutically acceptable salts.” Preparation of such salts is known to those skilled in pharmaceuticals. Examples of suitable pharmaceutically acceptable salts include, but are not limited to, hydrochlorides, bitartrates, acetates, palmitates, stearates, oleates, hydrobromides, sulfates, tartrates, citrates, maleates, and the like, or combinations of any of the foregoing.

In some suitable embodiments, the amine-containing compound is selected from the group consisting of dextromethorphan (e.g., dextromethorphan hydrobromide), cyclobenzaprine (e.g., cyclobenzaprine hydrochloride), benztropine (e.g., benztropine mesylate) and combinations thereof. For certain embodiments, the amine-containing compound is dextromethorphan (particularly dextromethorphan hydrobromide). For certain embodiments, the amine-containing compound is cyclobenzaprine (particularly cyclobenzaprine hydrochloride). For certain embodiments, the amine-containing compound is benztropine (particularly benztropine mesylate).

### Salts of Cyclic Organic Acids

Compositions of the present invention include one or more pharmaceutically acceptable salts of a non-NSAID cyclic organic acid compound (referred to herein as a “salt of a cyclic organic acid”). In this context, an “acid” is a compound that can be deprotonated in a neutral or low-pH environment to form an anion. Preferably, in this context, an acid is a compound with a pKa of no greater than 7, more preferably no greater than 5, and even more preferably no greater than 3.

Generally (but not necessarily), the salt of the cyclic organic acid will be pharmacologically inert. Surprisingly, in the practice of the present invention, such salts (but not the free acids) provide compositions with zero-order release kinetics with respect to the amine-containing compounds (including salts thereof).

Such cyclic organic acid compounds refer to compounds that include one or more cyclic groups. In this context, a “cyclic group” means a closed ring hydrocarbon group that is classified as an alicyclic group, aromatic group or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.). The term “heteroaryl group” means a mono- or polynuclear aromatic heterocyclic group.

Such cyclic organic acid compounds also include a functionality capable of being deprotonated to form an anion. Suitable functionalities can include, for example, a terminal carboxylate group, a sulfamate group, a sulfonate group, or a sulfimide group on the organic moiety. Other suitable salts of cyclic organic acid compounds include salts of vinylogous acids (e.g., ascorbic acid). The functional group responsible for the acidic/anionic characteristic may be included in the cyclic moiety, or may be included in an acyclic portion of the molecule. Preferred salts of cyclic organic compounds include a terminal carboxylate group. Other preferred salts include a sulfur-containing moiety, including sulfamate groups, sulfonate groups, or sulfimide groups.

Salts of cyclic organic acids used in compositions of the present invention preferably have a relatively low molecular weight. Preferably, the molecular weight is no greater than 1500, more preferably no greater than 1200, even more preferably no greater than 1000, even more preferably no greater than 800, and even more preferably no greater than 500 grams/mole (g/mol). Preferably, the molecular weight is at least 80, and more preferably at least 100 g/mol.

Preferred examples of such salts of a cyclic organic acid that are not NSAIDs include salts of the following acids: naphthoic acid, 1-hydroxy-2-naphthoic acid, 2-hydroxy-3-napththoic acid, pamoic acid, cyclamic acid, benzoic acid, and sulfimides of benzoic acid (including, for example, sodium benzoate and sodium saccharin), cinnamic acid, gentisic acid, vanillic acid, gallic acid, caffeic acid, ferulic acid, sinapic acid, lipoic acid, ascorbic acid, benzensulfonic acid, 4-acetamido-benzoic acid, (1S)-camphor-10-sulfonic acid, hippuric acid, lactobionic acid, mandelic acid, naphthalene sulfonates (including naphthalene-2-sulfonic acid and naphthalene-1,5,-disulfonic acid), nicotinic acid, orotic acid, L-pyroglutamic acid, and p-toluenesulfonic acid.

Salts of cyclic organic acids used in compositions of the present invention are pharmaceutically acceptable salts. Typically, such salts include metal salts, such as sodium, calcium, or potassium salts. Salts such as bismuth salts, magnesium salts, or zinc salts may also be suitable. Various combinations of counterions/salts can be used if desired. Salts are preferably the sodium and potassium salts of such acids, and more preferably, the sodium salts of such acids.

Particularly preferred salts of a cyclic organic acid that are not NSAIDs include: disodium pamoate, sodium saccharin, sodium cyclamate, sodium benzoate, sodium naphthoate, potassium benzoate, and combinations thereof. For certain embodiments, a particularly preferred salt of a cyclic organic acid that is not an NSAID is a pamoate (e.g., a disodium pamoate), Even more preferred salts include those shown below

| | Structure | Molecular Wt. | Comment |
|---|---|---|---|
| Disodium pamoate | 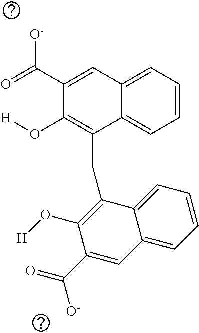 [SMILES: O=C([O-])c1cc2ccccc2c(Cc2c(O)c(C(=O)[O-])cc3ccccc23)c1O] | 432.22 | Used as an API counter ion (pharma- ceutical salt) |
| Sodium benzoate | 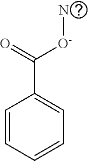 | 144.1 | Preserva- tive, lubricant |
| Sodium saccharin | 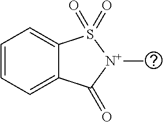 | 205.17 | Artificial sweetener |
| Sodium cyclamate | 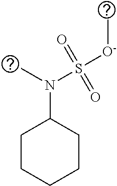 | 201.22 | Artificial sweetener |

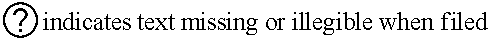

In preferred compositions, a salt of a cyclic organic acid is present in compositions in an amount to provide zero-order release kinetics under in vitro conditions. In particular, the sustained-release compositions of the present invention demonstrate a retarded rate of release, and preferably zero-order release, in the early stages (i.e., up to at least 50% total release, and preferably up to at least 60% total release) of a dissolution profile, as compared to a similar formulation that does not contain the salt of a cyclic organic acid.

Determination of such an amount is well within the capability of those skilled in the art, especially in light of the detailed disclosure provided herein. For example, a salt of a cyclic organic acid is present in a single dosage form of the current invention at an amount of 50 mg to 750 mg (for a twice per day dosage form). The skilled artisan will recognize that the molar ratio between the pharmacologically active amine-containing compound and the salt of the cyclic organic acid may be significant. In the practice of the present invention, the molar ratio is suitably in the range of 1:40 to 4:1, desirably in the range of 1:20 to 2:1, more desirably in the range of about 1:10 to 1:1, and even more desirably in the range of about 1:10 to 1:2.

### Pharmaceutically Acceptable Anionic Surfactants

Suitable pharmaceutically acceptable anionic surfactants include, for example, monovalent alkyl carboxylates, acyl lactylates, alkyl ether carboxylates, N-acyl sarcosinates, polyvalent alkyl carbonates, N-acyl glutamates, fatty acid-polypeptide condensates, sulfur-containing surfactants (e.g., sulfuric acid esters, alkyl sulfates such as sodium lauryl sulfate (SLS), ethoxylated alkyl sulfates, ester linked sulfonates such as docusate sodium or dioctyl sodium succinate (DSS), and alpha olefin sulfonates), and phosphated ethoxylated alcohols. Preferred surfactants are on the GRAS (“Generally Recognized as Safe”) list. Various combinations of pharmaceutically acceptable anionic surfactants can be used if desired.

In certain embodiments, the pharmaceutically acceptable anionic surfactant is a sulfur-containing surfactant, and particularly an alkyl sulfate, an ester-linked sulfonate, and combinations thereof. Preferred pharmaceutically acceptable anionic surfactants include sodium lauryl sulfate, docusate (i.e., dioctyl sulfosuccinate) sodium, docusate calcium, and combinations thereof. A particularly preferred anionic surfactant is docusate sodium. The structures of docusate sodium and sodium lauryl sulfate are as follows:

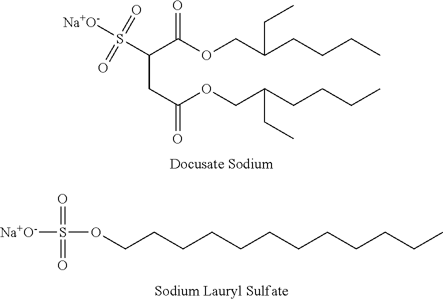

In preferred embodiments, a pharmaceutically acceptable anionic surfactant is present in compositions of the present invention in a release-modifying amount. A wide range of amounts can be used to tailor the rate and extent of release. Determination of such an amount is well within the capability of those skilled in the art, especially in light of the detailed disclosure provided herein.

In some embodiments, certain surfactants such as docusate can function as a stool softener when used at a therapeutic level; however, sub-therapeutic amounts can be used for release modification.

Such surfactants can be used for their abuse deterrence effects. For example, a surfactant could function as a nasal irritant, which would make crushing and inhaling the compositions undesirable. Also, a mixture of an amine-containing compound and a surfactant (e.g., docusate) in a hydrophilic matrix is difficult to extract and separate into the individual components, and injection of the mixture is undesirable and/or unsafe.

### Hydrophilic Matrix and Other Excipients

Compositions of the present invention include a hydrophilic matrix, wherein the pharmacologically active amine-containing compound (including salts thereof), the salt of a cyclic organic acid, and the optional anionic surfactant are within (e.g., mixed within) the hydrophilic matrix. Such matrix preferably includes at least one hydrophilic polymeric compound. The hydrophilic polymeric compound preferably forms a matrix that releases the amine-containing compound (e.g., opioid analgesic), which may be in the form of a pharmaceutically acceptable salt thereof, at a sustained rate upon exposure to liquids. That is, a hydrophilic matrix refers to a “gel forming” or “hydrogel” material wherein upon administration and exposure to liquids the hydrophilic matrix slowly expands to form a gel. The rate of release of the amine-containing compound from the hydrophilic matrix typically depends, at least in part, on the compound's partition coefficient between the components of the hydrophilic matrix and the aqueous phase within the gastrointestinal tract.

In a preferred tablet form, the hydrophilic matrix partially hydrates on the tablet surface to form a gel layer. The rate of hydration and gelling of the tablet surface affects the drug release from the tablet and contributes significantly to the sustained-release aspect of such products.

The sustained-release composition generally includes at least one hydrophilic polymeric compound in an amount of 10% to 90% by weight, preferably in an amount of 20% to 80% by weight, based on the total weight of the composition. In some embodiments, the hydrophilic polymeric compound is present in an amount of 10% to 50% by weight, or in an amount of 20% to 40% by weight, based on the total weight of the composition.

The hydrophilic polymeric component may be any known in the art. Exemplary hydrophilic polymeric components include gums, cellulose ethers, acrylic resins, polyvinyl pyrrolidone, protein-derived compounds, and combinations thereof. Exemplary gums include heteropolysaccharide gums and homopolysaccharide gums, such as xanthan, tragacanth, pectins, acacia, karaya, alginates, agar, guar, hydroxypropyl guar, carrageenan, locust bean gums, and gellan gums. Exemplary cellulose ethers include hydroxyalkyl celluloses and carboxyalkyl celluloses. Preferred cellulose ethers include hydroxyethyl celluloses, hydroxypropyl celluloses, hydroxypropyl methylcelluloses, carboxy methylcelluloses, and mixtures thereof. Exemplary acrylic resins include polymers and copolymers of acrylic acid, methacrylic acid, methyl acrylate, and methyl methacrylate. Various combinations of hydrophilic components can be used for various effects.

In some embodiments, the hydrophilic component is preferably a cellulose ether. Exemplary cellulose ethers include those commercially available under the trade designation METHOCEL Premium from Dow Chemical Co. Such methylcellulose and hypromellose (i.e., hydroxypropyl methylcellulose) products are a broad range of water-soluble cellulose ethers that enable pharmaceutical developers to create formulas for tablet coatings, granulation, sustained release, extrusion, and molding. For certain embodiments, the cellulose ether comprises a hydroxypropyl methylcellulose.

In preferred embodiments, hydroxypropyl methylcellulose is used in a composition having a tablet form. It is present throughout the tablet and partially hydrates on the tablet surface to form a gel layer. Overall dissolution rate and pharmacological agent availability are dependent on the rate of soluble pharmacologic agent diffusion through the wet gel and the rate of tablet erosion.

Hydroxypropyl methylcelluloses vary in their viscosity, methoxy content, and hydroxypropoxyl content. Hydroxypropyl methylcellulose with substitution rates of about 7-30% for the methoxyl group and greater than 7% or about 7-20% for the hydroxypropoxyl group are preferred for formation of this gel layer. More preferred are substitution rates of 19-30% for the methoxyl group and 7-12% for the hydroxypropyl group.

Varying the types of cellulose ethers can impact the release rate. For example, varying the types of METHOCEL cellulose ethers, which have different viscosities of 2% solutions in water (METHOCEL K4M Premium hypromellose 2208 (19-24% methoxy content; 7-12% hydroxypropyl content; 3,000-5,600 cps of a 2% solution in water); METHOCEL K15M Premium hypromellose 2208 (19-24% methoxy content; 7-12% hydroxypropyl content; 11,250-21,000 cps of a 2% solution in water); and METHOCEL K100M Premium hypromellose 2208 (19-24% methoxy content; 7-12% hydroxypropyl content; 80,000-120,000 cps of a 2% solution in water)) can help tailor release rates.

Compositions of the present invention can also include one or more excipients such as lubricants, glidants, flavorants, coloring agents, stabilizers, binders, fillers, disintegrants, diluents, suspending agents, viscosity enhancers, wetting agents, buffering agents, control release agents, crosslinking agents, preservatives, and the like. Such compounds are well known in the art of drug release and can be used in various combinations.

One particularly useful excipient that can form at least a portion of a composition of the present invention is a binder that includes, for example, a cellulose such as microcrystalline cellulose. An exemplary microcrystalline cellulose is that available under the trade designation AVICEL PH (e.g., AVICEL PH-101, AVICEL PH-102, AVICEL PH-301, AVICEL PH-302, and AVICEL RC-591) from FMC BioPolymers. The sustained-release composition optionally includes at least one microcrystalline cellulose in an amount of 3 wt-% to 50 wt-%, based on the total weight of the composition. For the practice of the present invention, however, AVICEL and other non-gelling microcrystalline cellulose components are not considered to provide the required “hydrophilic matrix” component.

Other additives can be incorporated into compositions of the present invention to further modify the rate and extent of release. For example, a non-pharmacologically active amine, such as tromethamine, triethanolamine, betaine, benzathine, or erbumine could be included in the compositions of the present invention to further modify the release rate.

Compositions of the present invention can optionally include compounds that function as abuse deterrents. For example, opioid antagonists (e.g., naltrexone, N-methylnaltrexone, naloxone) can be combined with opioid agonists to deter parenteral abuse of opioid agonists. Such opioid agonist/antagonist combinations can be chosen such that the opioid agonist and opioid antagonist are only extractable from the dosage form together, and at least a two-step extraction process is required to separate the opioid antagonist from the opioid agonist. The amount of opioid antagonist is sufficient to counteract opioid effects if extracted together and administered parenterally and/or the amount of antagonist is sufficient to cause the opioid agonist/antagonist combination to provide an aversive effect in a physically dependent human subject when the dosage form is orally administered. Typically, such compositions are formulated in such a way that if the dosage form is not tampered with, the antagonist passes through the GI tract intact; however, upon crushing, chewing, dissolving, etc., the euphoria-curbing antagonist is released.

In a similar fashion, compounds that cause nausea could be added to the formulation to prevent abusers from taking more than the intended dose. These components are added to the formulation at sub-therapeutic levels, such that no adverse effects are realized when the correct dose is taken.

Also, compositions of the present invention can include an aversive agent such as a dye (e.g., one that stains the mucous membrane of the nose and/or mouth) that is released when the dosage form is tampered with and provides a noticeable color or dye which makes the act of abuse visible to the abuser and to others such that the abuser is less likely to inhale, inject, and/or swallow the tampered dosage form. Examples of various dyes that can be employed as the aversive agent, including for example, and without limitation, FD&C Red No. 3, FD&C Red No. 20, FD&C Yellow No. 6, FD&C Blue No. 1, FD&C Blue No. 2, FD&C Green No. 1, FD&C Green No. 3, FD&C Green No. 5, FD&C Red No. 30, D&C Orange No. 5, D&C Red No. 8, D&C Red No. 33, caramel, and ferric oxide, red, other FD&C dyes and lakes, and natural coloring agents such as grape skin extract, beet red powder, beta-carotene, annato, carmine, turmeric, paprika, and combinations thereof.

The sustained-release compositions of the present invention may also include one or more hydrophobic polymers. The hydrophobic polymers may be used in an amount sufficient to slow the hydration of the hydrophilic matrix without disrupting it. For example, the hydrophobic polymer may be present in an amount of 0.5% to 20% by weight, based on the total weight of the composition.

Exemplary hydrophobic polymers include alkyl celluloses (e.g., C₁₋₆ alkyl celluloses, carboxymethylcellulose, ethylcellulose), other hydrophobic cellulosic materials or compounds (e.g., cellulose acetate phthalate, hydroxypropyl-methylcellulose phthalate), polyvinyl acetate polymers (e.g., polyvinyl acetate phthalate), polymers or copolymers derived from acrylic and/or methacrylic acid esters, zein, waxes (e.g., carnauba wax), shellac, hydrogenated vegetable oils, and combinations thereof.

### Pharmaceutical Compositions

Pharmaceutical compositions of the present invention are single dosage forms that can be in a form capable of providing sustained release of the amine-containing compound (which can be in the form or a salt). Herein, a “single dosage form” refers to the components of the composition be included within one physical unit (e.g., one tablet), whether it be in a uniform matrix, a multilayered construction, or some other configuration. Most commonly, this includes a tablet, which can include molded tablets, compressed tablets, or freeze-dried tablets. Other possible solid forms include pills, pellets, particulate forms (e.g., beads, powders, granules), and capsules (e.g., with particulate therein).

A single dosage form can be a coated dosage form with, for example, an outer layer of an immediate-release (IR) material (e.g., an amine-containing compound such as an opioid, a salt of a cyclic organic acid, or both, a release-modifying agent, a film coating for taste masking or for ease of swallowing, or the like), with a sustained-release (SR) core. Typically, such coated formulations do not demonstrate zero-order release kinetics during the initial immediate-release phase, but preferably demonstrate zero-order release kinetics during the dissolution of the sustained-release core.

A single dosage form can be incorporated into a multi-layered dosage form (e.g., tablet). For example, a bilayer tablet could be formulated to include a layer of a conventional immediate-release matrix and a layer of a sustained-release composition of the present invention. Optionally, a multi-layered dosage form could be coated.

Pharmaceutical compositions for use in accordance with the present invention may be formulated in a conventional manner to incorporate one or more physiologically acceptable carriers comprising excipients and auxiliaries. Compositions of the invention may be formulated as tablets, pills, capsules, and the like, for oral ingestion by a patient to be treated.

In certain preferred embodiments, the compositions of the present invention are formulated as tablets. Tablets have several advantages, particularly over capsules. For some drugs, it is recommended that the patient begin taking a smaller dose and gradually over time increase the dose to the desired level. This can help avoid undesirable side effects. Also, tablets can be preferable to capsules in this regard because a scored tablet easily can be broken to form a smaller dose. In addition, tableting processes such as wet granulation are generally simpler and less expensive than bead coating and capsule formation. Further, tablets can be safer to use because they may be less subject to tampering.

Pharmaceutical compositions of the present invention may be manufactured in a manner that is itself known, e.g., by means of conventional mixing, granulating, encapsulating, entrapping, or tabletting processes.

Pharmaceutical compositions suitable for use in the present invention include compositions where the ingredients are contained in an amount effective to achieve its intended purpose. The exact formulation, route of administration, and dosage for the pharmaceutical compositions of the present invention can be chosen by the individual physician in view of the patient's condition. (See, e.g., Fingl et al. in “The Pharmacological Basis of Therapeutics”, Ch. 1, p. 1 (1975)). The exact dosage will be determined on a drug-by-drug basis, in most cases. Dosage amount and interval may be adjusted individually to provide plasma levels of the active ingredients/moieties that are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. The amount of composition administered will, of course, be dependent on the subject being treated, on the subject's weight, the severity of the pain or other condition, the manner of administration, and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert.

It will be understood by those of skill in the art that numerous and various modifications can be made without departing from the spirit of the present invention. Therefore, it should be clearly understood that the forms of the present invention are illustrative only and are not intended to limit the scope of the present invention.

### EMBODIMENTS

Exemplary embodiments of the present invention include the following:

1. A sustained-release oral pharmaceutical composition comprising within a single dosage form:
a hydrophilic matrix;
a pharmacologically active amine-containing compound; and
a pharmaceutically acceptable salt of a non-NSAID cyclic organic acid compound;
wherein the amine-containing compound and the salt of the cyclic organic acid are within the hydrophilic matrix; and
wherein the composition exhibits a release profile of the amine-containing compound comprising a substantial portion that is representative of zero-order release kinetics under in vitro conditions.
   2. A sustained-release oral pharmaceutical composition comprising within a single dosage form:
a hydrophilic matrix;
a pharmacologically active amine-containing compound;
a pharmaceutically acceptable salt of a non-NSAID cyclic organic acid compound; and
a pharmaceutically acceptable anionic surfactant;
wherein the amine-containing compound, the salt of the cyclic organic acid, and the anionic surfactant are within the hydrophilic matrix.
   3. The composition of embodiment 2 which exhibits a release profile of the amine-containing compound comprising a substantial portion that is representative of zero-order release kinetics under in vitro conditions.
   4. The composition of any one of embodiments 1 through 3 wherein the amine group comprises a secondary amine, a tertiary amine, a primary amine, or combination thereof.
   5. The composition of embodiment 4 wherein the amine-containing compound comprises a tertiary amine.
   6. The composition of any one of embodiments 1 through 5 wherein the amine-containing compound is an opioid.
   7. The composition of embodiment 6 wherein the opioid is selected from the group consisting of morphine, codeine, hydromorphone, hydrocodone, oxycodone, oxymorphone, desomorphine, diacetylmorphine, buprenorphine, dihydrocodeine, nicomorphine, benzylmorphine, fentanyl, methadone, tramadol, propoxyphene, levorphanol, meperidine, and combinations thereof.
   8. The composition of embodiment 6 or embodiment 7 wherein the opioid is present in a pain-reducing amount.
   9. The composition of any one of embodiments 1 through 5 wherein the amine-containing compound is a non-opioid amine-containing compound.
   10. The composition of embodiment 9 wherein the non-opioid amine-containing compound is selected from the group consisting of dextromethorphan, cyclobenzaprine, benztropine, baclofen, arbaclofen, ritodrine, tizanidine, flurazepam, chlorpheniramine, doxylamine, diphenhydramine, diltiazem, rimantadine, amantadine, memantine, and combinations thereof.
   11. The composition of any one of the preceding embodiments wherein the amine-containing compound is a salt comprising a hydrochloride, a bitartrate, an acetate, a naphthylate, a tosylate, a mesylate, a besylate, a succinate, a palmitate, a stearate, an oleate, a pamoate, a laurate, a valerate, a hydrobromide, a sulfate, a methane sulfonate, a tartrate, a citrate, a maleate, or a combination of the foregoing.
   12. The composition of any one of the preceding embodiments wherein the salt of the cyclic organic acid is selected from the group consisting of disodium pamoate, sodium saccharin, sodium cyclamate, sodium benzoate, sodium naphthoate, potassium benzoate, and combinations thereof.
   13. The composition of any one of the preceding embodiments wherein the salt of the cyclic organic acid is a pamoate salt.
   14. The composition of any one of embodiments 2 through 13, as they depend on embodiment 2, wherein the pharmaceutically acceptable anionic surfactant is selected from the group consisting of monovalent alkyl carboxylates, acyl lactylates, alkyl ether carboxylates, N-acyl sarcosinates, polyvalent alkyl carbonates, N-acyl glutamates, fatty acid-polypeptide condensates, sulfur-containing surfactants, phosphated ethoxylated alcohols, and combinations thereof.
   15. The composition of any one of the preceding embodiments wherein the salt of the cyclic organic acid is present in an amount effective to provide zero-order release kinetics under in vitro conditions.
   16. The composition of any one of the preceding embodiments wherein the pharmaceutically acceptable anionic surfactant is present in a release-modifying amount.
   17. The composition of any one of the preceding embodiments wherein the single dosage form is a tablet form.
   18. The composition of any one of the previous embodiments wherein the hydrophilic matrix comprises at least one hydrophilic polymeric compound selected from the group consisting of a gum, a cellulose ether, an acrylic resin, a polyvinyl pyrrolidone, a protein-derived compound, and combinations thereof.
   19. The composition of embodiment 18 wherein the hydrophilic matrix comprises a cellulose ether.
   20. A method of providing a desired effect in a subject, the method comprising administering to a subject a composition of any one of embodiments 1 through 19.
   21. The method of embodiment 20 wherein providing the desired effect comprises preventing, alleviating, or ameliorating the level of pain in a subject.
   22. The method of embodiment 20 wherein providing the desired effect comprises suppressing cough.
   23. The method of any one of embodiments 20 through 22 wherein administering the composition comprises administering once or twice per day.
   24. The method of embodiment 23 wherein administering the composition comprises administering once per day.
   25. A method of preventing, alleviating, or ameliorating the level of pain in a subject, the method administering to a subject a composition comprising:
a hydrophilic matrix;
a pain-reducing amount of an opioid analgesic; and
a pharmaceutically acceptable salt of a non-NSAID cyclic organic acid compound present in an amount effective to provide zero-order release kinetics under in vitro conditions;
wherein the opioid analgesic and the salt of the cyclic organic acid are within the hydrophilic matrix; and
wherein the composition has a release profile comprising a substantial portion that is representative of zero-order release kinetics under in vitro conditions.
   26. A method of preventing, alleviating, or ameliorating the level of pain in a subject, the method administering to a subject a composition comprising:
a hydrophilic matrix;
a therapeutically effective amount of an opioid analgesic;
a pharmaceutically acceptable salt of a non-NSAID cyclic organic acid compound; and
a pharmaceutically acceptable anionic surfactant;
wherein the opioid analgesic, the salt of the cyclic organic acid, and the anionic surfactant are within the hydrophilic matrix.

### EXAMPLES

Objects and advantages of this invention are further illustrated by the following examples, but the particular materials and amounts thereof recited in these examples, as well as other conditions and details, should not be construed to unduly limit this invention.

### Example 1

### Preparation of Sustained Release Hydrophilic Matrix Tablets Containing Dextromethorphan Hydrobromide (DXM), Disodium Pamoate and Docusate Sodium (DSS) at Bench Top Scale

Each hydrophilic matrix tablet lot was produced by dry-blending the active substance(s) and excipients together followed by direct compression. The DXM (dextromethorphan hydrobromide) and disodium pamoate (when present) were added together with all excipients. Blending was accomplished using a GlobePharma “MiniBlend” blender (10 minutes at 28 rpm). Aliquots of the blend were massed out using an analytical balance and were compressed using a Manesty DC16 press. Each tablet aliquot was added to the die manually and compressed at a speed of 3 rpm (Prototype 1-3 was compressed at 10 rpm). Lots containing pamoate were compressed using 0.3750 inch (in.) round, concave Natoli tooling (HOB #91380). Lots without pamoate were compressed using 0.3125 in. round, concave Natoli tooling (HOB #91300). The compression force was varied until a target tablet breaking force of 14-16 kP was consistently achieved.

TABLE 1
Prototype formulation compositions (mg/tablet)

| | Formulation (mg/tablet) | | | | | Total |
|---|---|---|---|---|---|---|
| Prototype | Dextromethorphan | Methocel | Avicel | Disodium | Granular Docusate | Tablet Mass |
| No. | Hydrobromide | K4M | PH-302 | Pamoate | Sodium | (mg) |
| 1-1 | 15.0 | 120.1 | 45.0 | 109.9 | 17.1 | 307.1 |
| 1-2 | 15.0 | 120.1 | 45.0 | 219.8 | 17.1 | 417.0 |
| 1-3 | 15.0 | 120.1 | 45.0 | 439.6 | 17.1 | 636.8 |
| 1-4 | 15.0 | 120.1 | 45.0 | 219.8 | 0.0 | 399.9 |
| 1-5 | 15.0 | 120.1 | 45.0 | 0.0 | 0.0 | 180.1 |
| (Control) | | | | | | |

TABLE 2
Suppliers for tablet components

| Component | Vendor |
|---|---|
| Dextromethorphan Hydrobromide | Wockhardt Limited |
| Methocel K4M | Dow Chemical |
| Avicel PH-302 | FMC Biopolymer |
| Disodium Pamoate | Acros Organics |
| Granular Docusate Sodium | Cytec |

USP Apparatus 2 was used for the dissolution testing of the prototype tablets produced. The dissolution samples were assayed for DXM using HPLC with UV detection at 280 nm. The system parameters for both the chromatographic and dissolution analysis are shown below.
System: Agilent 1100 Series HPLC System
Column: Phenomenex Jupiter C18, 250×4.6 mm ID, 5μ, 300 Å Part No.: 00G-4053-EO
Detector: UV detector, 280 nm
Mobile Phase A: 94.7/5.0/0.3 (v/v/v) water/methanol/TFA
Mobile Phase B: Pure methanol
Method Type Gradient
Flow Rate: 1.5 mL/min
Injection Volume: 30 μL
Run Time: 14.00 minutes (12.01-14.00 minutes is reequilibration)
Peakwidth: >0.1 min
Column Temperature: 35° C.
Autosampler temp: Ambient

TABLE 3
Gradient profile for HPLC mobile phases A and B

| | | |
|---|---|---|
| Initial | 95% A | 5% B |
| 9.00 min. | 0% A | 100% B |
| 12.00 min. | 0% A | 100% B |
| 12.01 min. | 95% A | 5% B |
| 14.00 min. | 95% A | 5% B |

TABLE 4
Dissolution parameters

| Parameters | Requirements |
|---|---|
| Method Type | USP Apparatus 2 (Paddle Method) |
| Rotation Speed | 50 rpm |
| Dissolution Media | pH 7.5 phosphate buffer (0.05M, potassium |
| | phosphate monobasic 0.68%/NaOH 0.164%) |
| Media Volume | 900 mL |
| Media Temperature | 37.0 ± 0.5 C. |
| Sampling Time Points | 1, 3, 6, 9, 12, 18 and 24 hours |
| Sampling Volume | 8 mL without media replacement (Use |
| | 35 μm Filter discs, QLA, part number |
| | FIL035-01-a) |

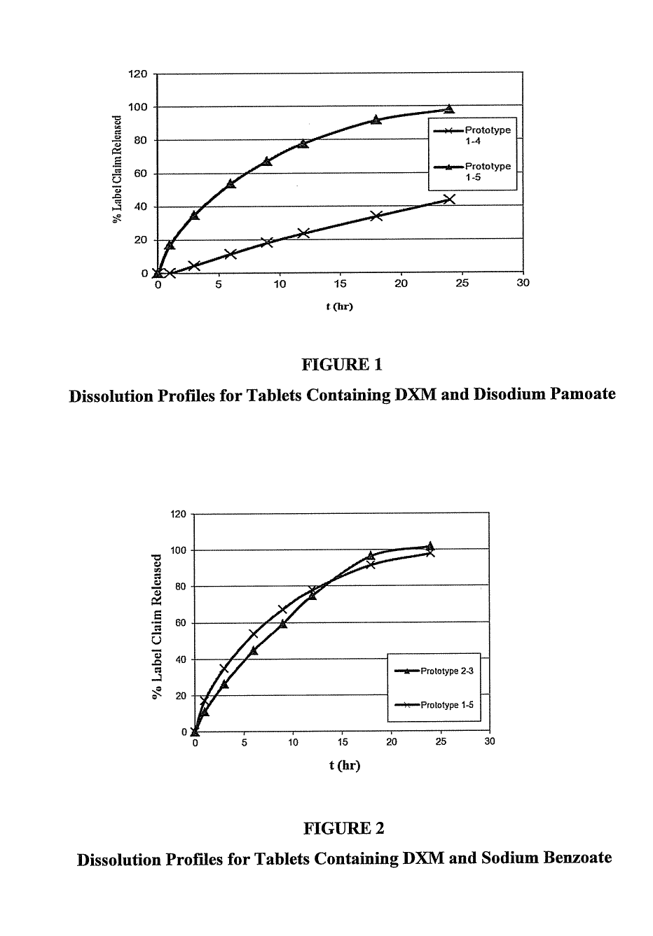

Prototype 1-4 in FIG. 1 shows a release profile for pamoate and DXM without the addition of docusate to the formulation. A rate of release similar to Prototype 1-2 was seen (Prototype 1-2 plot not shown). This demonstrated that the impact of docusate sodium (at this specific level) to retard the rate of release may be less than has been seen for other formulations (not shown here).

Prototype 1-5 was a Control. This was a typical matrix tablet that contained neither the pamoate nor the docusate. First-order release kinetics was demonstrated for the Control.

The remaining formulations (Prototypes 1-1 and 1-3, plots not shown) demonstrated the effect of varying levels of disodium pamoate for tablets that contained constant docusate and constant DXM. The results showed that release rates can be adjusted with different pamoate levels in the formulation. For these formulations higher levels of pamoate increase the rate of release. Prototype 1-3 contained an extremely high level of pamoate that impacted the integrity of the gel layer. A more rapid rate of release was seen with a deviation from zero-order kinetics; thus, this prototype may contain pamoate at a level higher than the limit for effective control of zero-order release.

The extent of zero-order behavior can be quantified in terms of a linear regression fit. A formulation exhibiting perfect zero-order kinetics would have an r² value of 1.00. For the pamoate samples the formulations that were closest to achieving theoretical zero-order behavior were prototypes 1-2 and 1-4. Their linear regression r² values are 0.9953 and 0.9949 respectively. In contrast the Control (Prototype 1-5) had an r² value of 0.8760 demonstrating poor correlation to a simple linear fit model.

### Example 2

### Preparation of Sustained Release Hydrophilic Matrix Tablets Containing Dextromethorphan Hydrobromide (DXM), Sodium Benzoate and Docusate Sodium (DSS) at Bench Top Scale

Each hydrophilic matrix tablet lot was produced by dry-blending the active substance(s) and excipients together followed by direct compression. The DXM and sodium benzoate (when present) were added together with all excipients. Blending was accomplished using a GlobePharma “MiniBlend” blender (10 minutes at 28 rpm). Aliquots of the blend were massed out using an analytical balance and were compressed using a Manesty DC16 press. Each tablet aliquot was added to the die manually and compressed at a speed of 3 rpm. Lots were compressed using 0.3750 in. round, concave Natoli tooling (HOB #91380). The control was compressed using 0.3125 in. round, concave Natoli tooling (HOB #91300). The compression force was varied until a target tablet breaking force of 14-16 kP was consistently achieved.

TABLE 5
Prototype formulation compositions (mg/tablet)

| | Formulation (mg/tablet) | | | | | Total |
|---|---|---|---|---|---|---|
| Prototype | Dextromethorphan | Methocel | Avicel | Sodium | Granular Docusate | Tablet Mass |
| No. | Hydrobromide | K4M | PH-302 | Benzoate | Sodium | (mg) |
| 2-1 | 15.0 | 120.1 | 45.0 | 109.9 | 17.1 | 307.1 |
| 2-2 | 15.0 | 120.1 | 45.0 | 219.8 | 17.1 | 417.0 |
| 2-3 | 15.0 | 120.1 | 45.0 | 219.8 | 0.0 | 399.9 |
| 1-5 | 15.0 | 120.1 | 45.0 | 0.0 | 0.0 | 180.1 |
| (Control) | | | | | | |

TABLE 6
Suppliers for tablet components

| Component | Vendor |
|---|---|
| Dextromethorphan Hydrobromide | Wockhardt Limited |
| Methocel K4M | Dow Chemical |
| Avicel PH-302 | FMC Biopolymer |
| Sodium benzoate | Riedel-de Haen |
| Granular Docusate Sodium | Cytec |

USP Apparatus 2 was used for the dissolution testing of the prototype tablets produced. The dissolution samples were assayed for DXM using HPLC with UV detection at 280 nm. The system parameters for both the chromatographic and dissolution analysis are shown in Example 1.

Referring to FIG. 2, Prototypes 2-1 and 2-2 (plots not shown) demonstrated the effects of increasing sodium benzoate for tablets that contained both constant DXM and constant Docusate. In this case increasing the amount of sodium benzoate increased the rate of release. Also, for both formulations the inclusion of docusate retarded the rate of release compared to formulations 2-3 and 1-5 that did not contain docusate. Formulations 2-1 and 2-2 exhibit essentially zero-order release out to 24 hours, though some slight curvature is seen. Interestingly, the binary system containing sodium benzoate and DXM (Prototype 2-3, contains no docusate) had a release profile indicative of zero-order release out to 18 hours.

Prototype 1-5 was a Control. This was a typical matrix tablet that contained neither the sodium benzoate nor the docusate sodium. First-order release kinetics was demonstrated for the Control.

### Example 3

### Preparation of Sustained Release Hydrophilic Matrix Tablets Containing Dextromethorphan Hydrobromide (DXM), Sodium Cyclamate and Docusate Sodium (DSS) at Bench Top Scale

Each hydrophilic matrix tablet lot was produced by dry-blending the active substance(s) and excipients together followed by direct compression. The DXM and sodium cyclamate (when present) were added together with all excipients. Blending was accomplished using a GlobePharma “MiniBlend” blender (10 minutes at 28 rpm). Aliquots of the blend were massed out using an analytical balance and were compressed using a Manesty DC16 press. Each tablet aliquot was added to the die manually and compressed at a speed of 3 rpm. Prototype 3-1 was compressed using 0.3125 in. round, concave Natoli tooling (HOB #91300). Prototypes 3-2 and 3-3 were compressed using 0.3750 in. round, concave Natoli tooling (HOB #91380). The control formulation was compressed using 0.3125 in. round, concave Natoli tooling (HOB #91300). The compression force was varied until a target tablet breaking force of 14-16 kP was consistently achieved.

TABLE 7
Prototype formulation compositions (mg/tablet)

| | Formulation (mg/tablet) | | | | | Total |
|---|---|---|---|---|---|---|
| Prototype | Dextromethorphan | Methocel | Avicel | Sodium | Granular Docusate | Tablet Mass |
| No. | Hydrobromide | K4M | PH-302 | Cyclamate | Sodium | (mg) |
| 3-1 | 15.0 | 120.1 | 45.0 | 109.9 | 17.1 | 307.1 |
| 3-2 | 15.0 | 120.1 | 45.0 | 219.8 | 17.1 | 417.0 |
| 3-3 | 15.0 | 120.1 | 45.0 | 219.8 | 0.0 | 399.9 |
| 1-5 | 15.0 | 120.1 | 45.0 | 0.0 | 0.0 | 180.1 |
| (Control) | | | | | | |

TABLE 8
Suppliers for tablet components

| Component | Vendor |
|---|---|
| Dextromethorphan Hydrobromide | Wockhardt Limited |
| Methocel K4M | Dow Chemical |
| Avicel PH-302 | FMC Biopolymer |
| Sodium Cyclamate | Acros Organics |
| Granular Docusate Sodium | Cytec |

USP Apparatus 2 was used for the dissolution testing of the prototype tablets produced. The dissolution samples were assayed for DXM using HPLC with UV detection at 280 nm. The system parameters for both the chromatographic and dissolution analysis are shown in Example 1.

For the sodium cyclamate samples, little effect was seen on increasing cyclamate for the two samples with constant docusate (Prototypes 3-1 to and 3-2, plots not shown). Docusate sodium did have the effect of slowing release compared to formulations that did not contain docusate. The tablet without docusate released faster and had zero-order characteristics out to 18 hours (Prototype 3-3, FIG. 3). The three formulations were demonstrated to have zero-order characteristics out to 18 hours for Prototype 3-3 (FIG. 3), and 24 hours for Prototypes 3-1 and 3-2. Prototype 1-5 was a Control. This was a typical matrix tablet that contained neither the cyclamate nor the docusate. Characteristic first-order release was observed for the Control.

### Example 4

### Preparation of Sustained Release Hydrophilic Matrix Tablets Containing Dextromethorphan Hydrobromide (DXM), Sodium Saccharin and Docusate Sodium (DSS) at Bench Top Scale

Each hydrophilic matrix tablet lot was produced by dry-blending the active substance(s) and excipients together followed by direct compression. The DXM and sodium saccharin (when present) were added together with all excipients. Blending was accomplished using a GlobePharma “MiniBlend” blender (10 minutes at 28 rpm). Aliquots of the blend were massed out using an analytical balance and were compressed using a Manesty DC16 press. Each tablet aliquot was added to the die manually and compressed at a speed of 3 rpm. Prototype 4-1 was compressed using 0.3125 in. round, concave Natoli tooling (HOB #91300). Prototypes 4-2 and 4-3 were compressed using 0.3750 in. round, concave Natoli tooling (HOB #91380). The control formulation was compressed using 0.3125 in. round, concave Natoli tooling (HOB #91300). The compression force was varied until a target tablet breaking force of 14-16 kP was consistently achieved.

TABLE 9
Prototype formulation compositions (mg/tablet)

| | Formulation (mg/tablet) | | | | | Total |
|---|---|---|---|---|---|---|
| Prototype | Dextromethorphan | Methocel | Avicel | Sodium | Granular Docusate | Tablet Mass |
| No. | Hydrobromide | K4M | PH-302 | Saccharin | Sodium | (mg) |
| 4-1 | 15.0 | 120.1 | 45.0 | 109.9 | 17.1 | 307.1 |
| 4-2 | 15.0 | 120.1 | 45.0 | 219.8 | 17.1 | 417.0 |
| 4-3 | 15.0 | 120.1 | 45.0 | 219.8 | 0.0 | 399.9 |
| 1-5 | 15.0 | 120.1 | 45.0 | 0.0 | 0.0 | 180.1 |
| (Control) | | | | | | |

TABLE 10
Suppliers for tablets components

| Component | Vendor |
|---|---|
| Dextromethorphan Hydrobromide | Wockhardt Limited |
| Methocel K4M | Dow Chemical |
| Avicel PH-302 | FMC Biopolymer |
| Sodium Saccharin | Fisher |
| Granular Docusate Sodium | Cytec |

USP Apparatus 2 was used for the dissolution testing of the prototype tablets produced. The dissolution samples were assayed for DXM using HPLC with UV detection at 280 nm. The system parameters for both the chromatographic and dissolution analysis are shown in Example 1.

Referring to FIG. 4, the results for sodium saccharin were similar to those seen for sodium cyclamate. In the presence of all three components (DXM, saccharin and docusate) increasing levels of saccharin increased the rate of release (Prototypes 4-1 to 4-2, plots not shown), though zero-order release characteristics were maintained. Inclusion of docusate in formulations slowed the release of DXM. The binary system (Prototype 4-3) exhibited essentially linear release out to 18 hours. Similar results were seen for binary systems (no docusate) containing benzoate or cyclamate.

Prototype 1-5 was a Control. This was a typical matrix tablet that contained neither the saccharin nor the docusate. Characteristic first-order release was observed for the Control.

### Example 5

### Preparation of Comparative Hydrophilic Matrix Tablets Containing Dextromethorphan Hydrobromide (DXM), Sodium Citrate and Docusate Sodium (DSS) at Bench Top Scale

Each hydrophilic matrix tablet lot was produced by dry-blending the active substance(s) and excipients together followed by direct compression. The DXM and sodium citrate (when present) were added together with all excipients. Blending was accomplished using a GlobePharma “MiniBlend” blender (10 minutes at 28 rpm). Aliquots of the blend were massed out using an analytical balance and were compressed using a Manesty DC16 press. Each tablet aliquot was added to the die manually and compressed at a speed of 3 rpm. Lots were compressed using 0.3750 in. round, concave Natoli tooling (HOB #91380). The control formulation was compressed using 0.3125 in. round, concave Natoli tooling (HOB #91300). The compression force was varied until a target tablet breaking force of 14-16 kP was consistently achieved.

TABLE 11
Prototype formulation compositions (mg/tablet)

| Comparative | Formulation (mg/tablet) | | | | | Total |
|---|---|---|---|---|---|---|
| Prototype | Dextromethorphan | Methocel | Avicel | Sodium | Granular Docusate | Tablet Mass |
| No. | Hydrobromide | K4M | PH-302 | Citrate | Sodium | (mg) |
| 5-1 | 15.0 | 120.1 | 45.0 | 109.9 | 17.1 | 307.1 |
| 5-2 | 15.0 | 120.1 | 45.0 | 219.8 | 17.1 | 417.0 |
| 5-3 | 15.0 | 120.1 | 45.0 | 219.8 | 0.0 | 399.9 |
| 1-5 | 15.0 | 120.1 | 45.0 | 0.0 | 0.0 | 180.1 |
| (Control) | | | | | | |

TABLE 12
Suppliers for prototype tablet components

| Component | Vendor |
|---|---|
| Dextromethorphan Hydrobromide | Wockhardt Limited |
| Methocel K4M | Dow Chemical |
| Avicel PH-302 | FMC Biopolymer |
| Sodium citrate dihydrate | Fisher |
| Granular Docusate Sodium | Cytec |

USP Apparatus 2 was used for the dissolution testing of the prototype tablets produced. The dissolution samples were assayed for DXM using HPLC with UV detection at 280 nm. The system parameters for both the chromatographic and dissolution analysis are shown in Example 1.

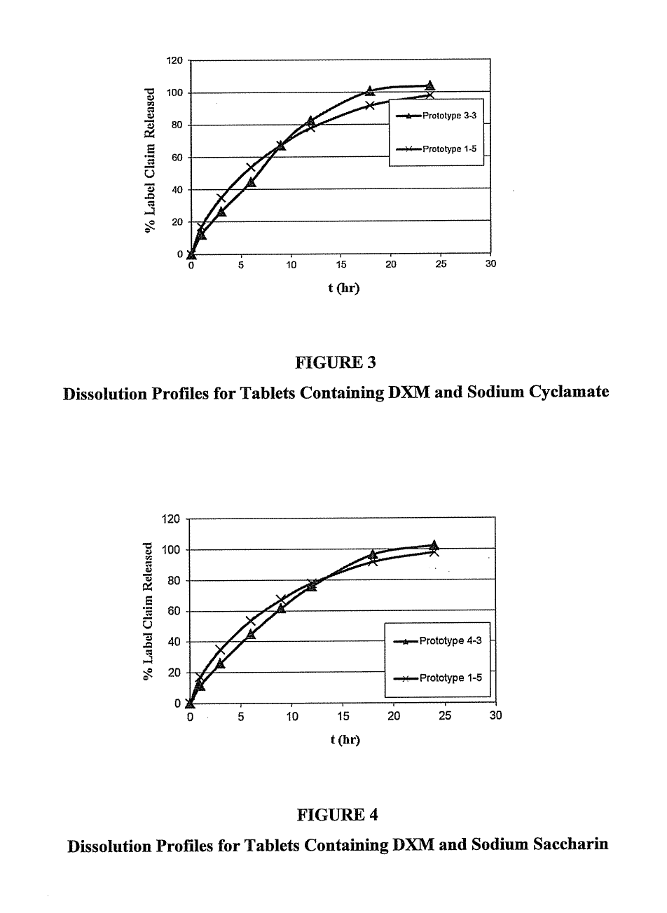

The results shown in FIG. 5 explore the effect of sodium citrate dihydrate for similar prototypes. Comparative Prototypes 5-1 and 5-2 (plots not shown) illustrated the effect of different levels of citrate in the presence of DXM and docusate. Increased citrate did increase the rate of release, but the docusate had an inhibitory effect for both formulations. In the absence of docusate, the citrate and DXM tablet released faster than the control (compare Comparative Prototypes 5-3 and 1-5).

The plots for citrate formulations all showed curvature and were indicative of 1^{st} order kinetics. For example, Comparative Prototype 5-1 (r² 0.9271, plot not shown), Comparative Prototype 5-2 (r² 0.9199, plot not shown), Comparative Prototype 5-3 (r² 0.7017), and Comparative Prototype 1-5 (control, r² 0.8760) all showed significant deviation from the linear fit model, where r² indicates the overall goodness of fit of the linear model.

### Example 6

### Preparation of Comparative Hydrophilic Matrix Tablets Containing Dextromethorphan Hydrobromide (DXM), Sodium Acetate and Docusate Sodium (DSS) at Bench Top Scale

Each hydrophilic matrix tablet lot was produced by dry-blending the active substance(s) and excipients together followed by direct compression. Prior to blending, the sodium acetate was ground in a mortar and pestle to achieve a fine, granular powder. The DXM and sodium acetate (when present) were added together with all excipients. Blending was accomplished using a GlobePharma “MiniBlend” blender (10 minutes at 28 rpm). Aliquots of the blend were massed out using an analytical balance and were compressed using a Manesty DC16 press. Each tablet aliquot was added to the die manually and compressed at a speed of 3 rpm. Prototype 6-1 was compressed using 0.3125 in. round, concave Natoli tooling (HOB #91300). Prototypes 6-2 and 6-3 were compressed using 0.3750 in. round, concave Natoli tooling (HOB #91380). The control formulation was compressed using 0.3125 in. round, concave Natoli tooling (HOB #91300). The compression force was varied until a target tablet breaking force of 14-16 kP was consistently achieved.

TABLE 13
Prototype formulation compositions (mg/tablet)

| Comparative | Formulation (mg/tablet) | | | | | Total |
|---|---|---|---|---|---|---|
| Prototype | Dextromethorphan | Methocel | Avicel | Sodium | Granular Docusate | Tablet Mass |
| No. | Hydrobromide | K4M | PH-302 | Acetate | Sodium | (mg) |
| 6-1 | 15.0 | 120.1 | 45.0 | 109.9 | 17.1 | 307.1 |
| 6-2 | 15.0 | 120.1 | 45.0 | 219.8 | 17.1 | 417.0 |
| 6-3 | 15.0 | 120.1 | 45.0 | 219.8 | 0.0 | 399.9 |
| 1-5 (control) | 15.0 | 120.1 | 45.0 | 0.0 | 0.0 | 180.1 |

TABLE 14
Suppliers for prototype tablets

| Component | Vendor |
|---|---|
| Dextromethorphan Hydrobromide | Wockhardt Limited |
| Methocel K4M | Dow Chemical |
| Avicel PH-302 | FMC Biopolymer |
| Sodium acetate trihydrate | EMD |
| Granular Docusate Sodium | Cytec |

USP Apparatus 2 was used for the dissolution testing of the prototype tablets produced. The dissolution samples were assayed for DXM using HPLC with UV detection at 280 nm. The system parameters for both the chromatographic and dissolution analysis are shown in Example 1.

The results shown in FIG. 6 demonstrated effects similar to those seen for the sodium citrate. Comparative Prototypes 6-1 and 6-2 (plots not shown) illustrated the effect of different levels of acetate in the presence of DXM and docusate. Increasing the level of acetate had negligible impact on the rate of release for the formulations that containined doscusate. As demonstrated with citrate-containing prototypes, docusate did slow down the rate of release with some curvature seen in the plots (indicative of a retarded 1^{st} order profile). In the absence of docusate, the acetate and DXM tablet released faster than the Control (compare Comparative Prototype 6-3 to Control 1-5). For all three acetate formulations, first-order release was seen.

### Example 7

### Preparation of Sustained Release Hydrophilic Matrix Tablets Containing Tramadol Hydrochloride (TMD), Disodium Pamoate and Docusate Sodium (DSS) at Bench Top Scale

Each hydrophilic matrix tablet lot was produced by dry-blending the active substance(s) and excipients together followed by direct compression. The TMD and disodium pamoate were added together with all excipients. Blending was accomplished using a GlobePharma “MiniBlend” blender (10 minutes at 28 rpm). Aliquots of the blend were massed out using an analytical balance and were compressed using a Manesty DC16 press. Each tablet aliquot was added to the die manually and compressed at a speed of 3 rpm. Prototypes 7-1, 7-2 and 7-3 were compressed using 0.3750 in. round, concave Natoli tooling (HOB #91380). The compression force was varied until a target tablet breaking force of 14-16 kP was consistently achieved.

TABLE 15
Prototype formulation compositions (mg/tablet)

| | Formulation (mg/tablet) | | | | | Total |
|---|---|---|---|---|---|---|
| Proto- | Tramadol | | | Di- | Granular | Tablet |
| type | Hydro- | Methocel | Avicel | sodium | Docusate | Mass |
| No. | chloride | K4M | PH-302 | Pamoate | Sodium | (mg) |
| 7-1 | 15.0 | 120.1 | 45.0 | 110.0 | 17.1 | 307.2 |
| 7-2 | 15.0 | 120.1 | 45.0 | 219.8 | 17.1 | 417.0 |
| 7-3 | 15.0 | 120.1 | 45.0 | 219.8 | 0.0 | 399.9 |

TABLE 16
Suppliers for prototype tablets

| Component | Vendor |
|---|---|
| Tramadol Hydrochloride | Spectrum Chemical Mfg. Corp. |
| Methocel K4M | Dow Chemical |
| Avicel PH-302 | FMC Biopolymer |
| Disodium Pamoate | Acros Organics |
| Granular Docusate Sodium | Cytec |

USP Apparatus 2 was used for the dissolution testing of the prototype tablets. The dissolution samples were assayed for TMD using HPLC with UV detection at 280 nm. The system parameters for both the chromatographic and dissolution analysis are shown in Example 1.

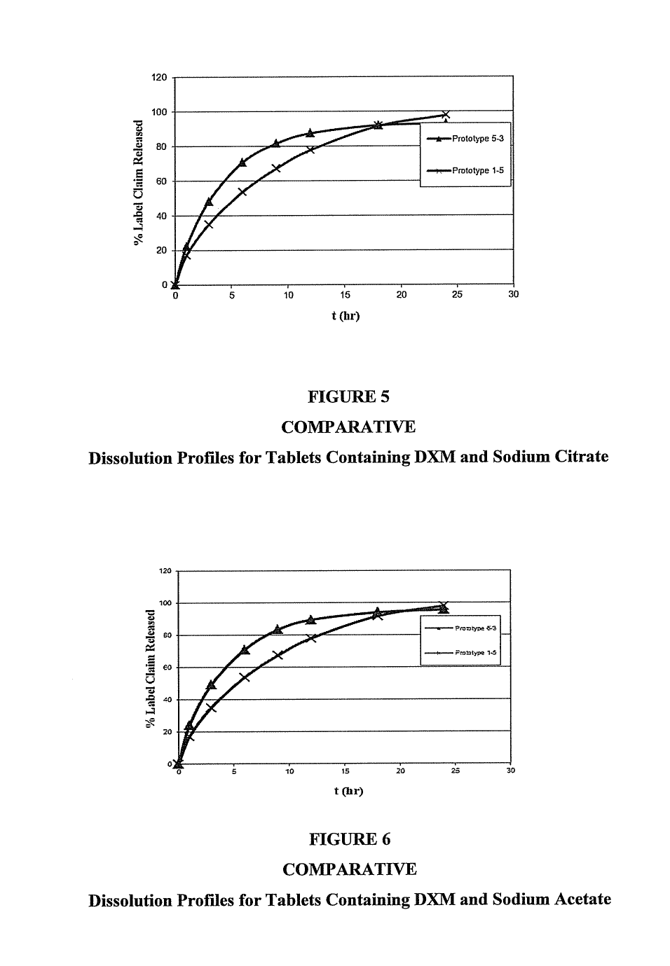

The results shown in FIG. 7 demonstrate the applicability of the present invention to the opioid class of drugs. For example, when tramadol (a synthetic opioid analgesic) was formulated in a matrix tablet with disodium pamoate, zero-order characteristics were seen out to 24 hours (see Prototype 7-3). The incorporation of docusate sodium into the remaining formulations showed that this excipient can be used to further adjust rates of release by slowing the rate of release For a constant level of docusate sodium, it was shown that varying the amount of disodium pamoate can vary the rate of release with a higher level of pamoate increasing the rate of tramadol release (for Prototypes 7-1 and 7-2, plots not shown). The r² values resulting from linear fits of the data by linear regression were: Prototype 7-1, r²=0.9873; Prototype 7-2, r²=0.9698; Prototype 7-3, r²=0.9676. These results demonstrated zero order release for formulations containing tramadol and pamoate as well as formulations containing tramadol, pamoate and docusate.

### Example 8

### Preparation of Comparative Hydrophilic Matrix Tablets Containing Tramadol Hydrochloride (TMD), Ibuprofen (Free Acid) and Docusate Sodium (DSS) at Bench Top Scale

Each hydrophilic matrix tablet lot was produced by dry-blending the active substance(s) and excipients together followed by direct compression. The TMD and ibuprofen (free acid) were added together with all excipients. Blending was accomplished using a GlobePharma “MiniBlend” blender (10 minutes at 28 rpm). Aliquots of the blend were massed out using an analytical balance and were compressed using a Manesty DC16 press. Each tablet aliquot was added to the die manually and compressed at a speed of 3 rpm. Prototypes 8-1, 8-2 and 8-3 were compressed using 0.3750 in. round, concave Natoli tooling (HOB #91380). The compression force was varied until a target tablet breaking force of 14-16 kP was consistently achieved.

TABLE 17
Prototype formulation compositions (mg/tablet)

| | Formulation (mg/tablet) | | | | | Total |
|---|---|---|---|---|---|---|
| Proto- | Tramadol | | | | Granular | Tablet |
| type | Hydro- | Methocel | Avicel | Ibuprofen | Docusate | Mass |
| No. | chloride | K4M | PH-302 | free acid | Sodium | (mg) |
| 8-1 | 15.0 | 120.1 | 45.0 | 219.8 | 0.0 | 399.9 |
| 8-2 | 15.0 | 120.1 | 45.0 | 219.8 | 17.1 | 417.0 |
| 8-3 | 15.0 | 120.1 | 45.0 | 220.0 | 118.0 | 518.1 |

TABLE 18
Suppliers for prototype tablets

| Component | Vendor |
|---|---|
| Tramadol Hydrochloride | Spectrum Chemical Mfg. Corp. |
| Methocel K4M | Dow Chemical |
| Avicel PH-302 | FMC Biopolymer |
| Ibuprofen FA | Acros Organics |
| Granular Docusate Sodium | Cytec |

USP Apparatus 2 was used for the dissolution testing of the prototype tablets. The dissolution samples were assayed for TMD using HPLC with UV detection at 280 nm. The system parameters for both the chromatographic and dissolution analysis are shown in Example 1.

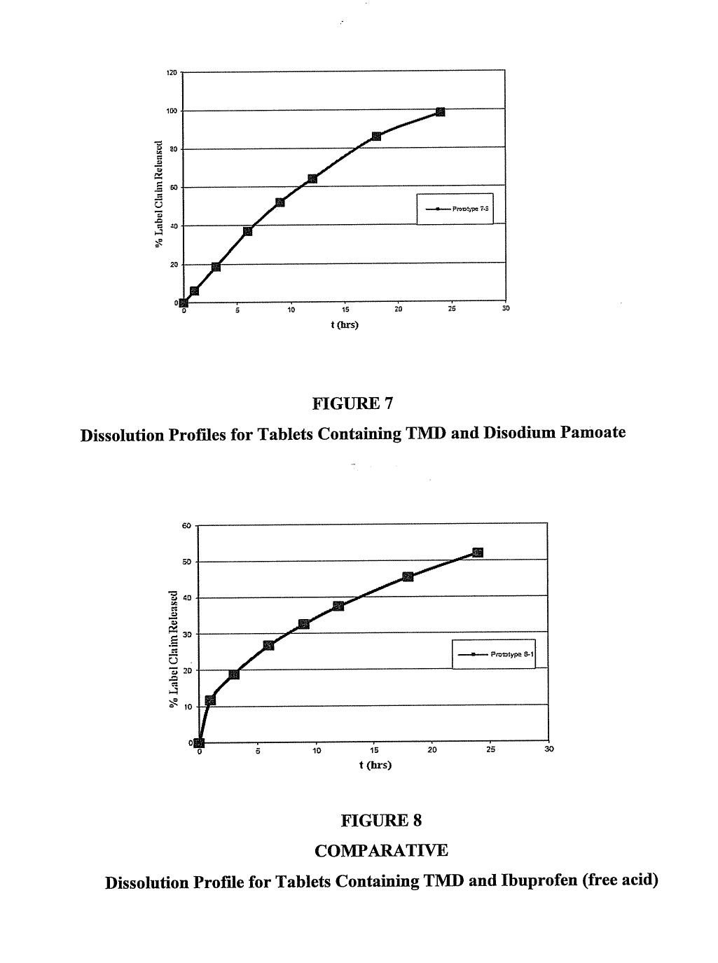

The results shown in FIG. 8 demonstrated the requirement for a pharmaceutically acceptable salt form of the anionic acid. The free acid form of ibuprofen used is not a salt in this case. For example, when tramadol (a synthetic opioid analgesic) was formulated in a matrix tablet with ibuprofen (free acid form), first order characteristics were seen out to 24 hours (see Prototype 8-1). The incorporation of docusate sodium into the remaining formulations (see Prototypes 8-2 and 8-3, plots not shown) showed that this excipient can be used to further adjust rates of release but in this case the curves indicated a retarded 1^{st} order release and not zero order release.

### Example 9

### Preparation of Sustained Release Hydrophilic Matrix Tablets Containing Dextromethorphan Hydrobromide (DXM), Potassium Benzoate and Docusate Sodium (DSS) at Bench Top Scale

Each hydrophilic matrix tablet lot was produced by dry-blending the active substance(s) and excipients together followed by direct compression. The DXM and potassium benzoate were added together with all excipients. Blending was accomplished using a GlobePharma “MiniBlend” blender (15 minutes at 28 rpm). Aliquots of the blend were massed out using an analytical balance and were compressed using a Manesty DC16 press. Each tablet aliquot was added to the die manually and compressed at a speed of 3 rpm. Prototypes 9-1, 9-2 and 9-3 were compressed using 0.3750 in. round, concave Natoli tooling (HOB #91380). The compression force was varied until a target tablet breaking force of 14-16 kP was consistently achieved.

TABLE 19
Prototype formulation compositions (mg/tablet)

| | Formulation (mg/tablet) | | | | | Total |
|---|---|---|---|---|---|---|
| Prototype | Dextromethorphan | Methocel | Avicel | Potassium | Granular Docusate | Tablet Mass |
| No. | Hydrobromide | K4M | PH-302 | Benzoate | Sodium | (mg) |
| 9-1 | 15.0 | 120.1 | 45.0 | 219.8 | 0.0 | 399.9 |
| 9-2 | 15.0 | 120.1 | 45.0 | 109.9 | 17.1 | 307.1 |
| 9-3 | 15.0 | 120.1 | 45.0 | 219.8 | 17.1 | 417.0 |
| 1-5 | 15.0 | 120.1 | 45.0 | 0.0 | 0.0 | 180.1 |
| (control) | | | | | | |

TABLE 20
Suppliers for prototype tablets

| Component | Vendor |
|---|---|
| Dextromethorphan Hydrobromide | Wockhardt |
| Methocel K4M | Dow Chemical |
| Avicel PH-302 | FMC Biopolymer |
| Potassium Benzoate | Alfa Aesar |
| Granular Docusate Sodium | Cytec |

USP Apparatus 2 was used for the dissolution testing of the prototype tablets. The dissolution samples were assayed for DXM using HPLC with UV detection at 280 nm. The system parameters for both the chromatographic and dissolution analysis are shown in Example 1.

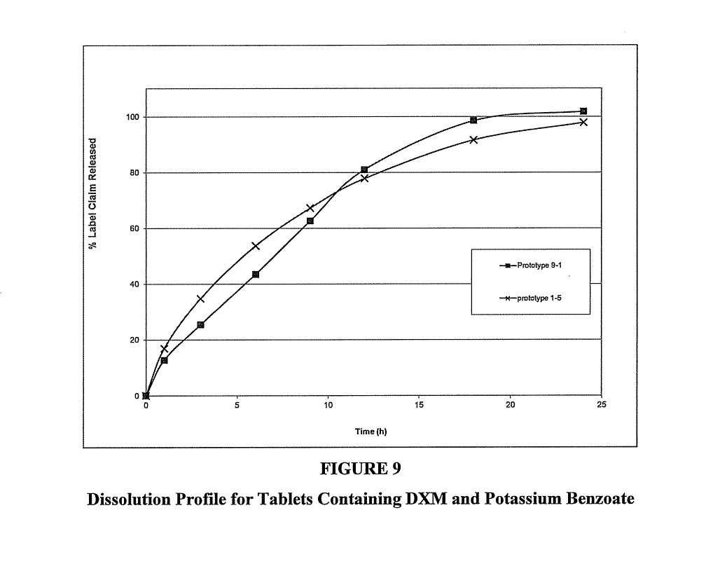

The results shown in FIG. 9 demonstrate that the salt of the anionic acid component (salt of the non-NSAID cyclic organic compound) is not limited to sodium salts. For example, when dextromethorphan hydrobromide is formulated in a matrix tablet with potassium benzoate, zero-order characteristics are seen out to 18 hours (see Prototype 9-1). The effect is demonstrated through comparison with the control formulation that exhibits 1^{st} order release kinetics. The incorporation of docusate sodium into the remaining formulations (9-2 and 9-3, plots not shown) demonstrates that this excipient can be used to further adjust rates of release.

The effect of docusate sodium is similar to that seen for other formulations where the presence of docusate retards the overall rate of release. Additionally, it was demonstrated that for a constant level of docusate sodium, varying the amount of potassium benzoate can vary the rate of release with a higher level of potassium benzoate increasing the rate of dextromethorphan release.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein.

1. A sustained-release oral pharmaceutical composition comprising within a single dosage form:
a hydrophilic matrix;
a pharmacologically active amine-containing compound; and
a pharmaceutically acceptable salt of a non-NSAID cyclic organic acid compound;
wherein the amine-containing compound and the salt of the cyclic organic acid are within the hydrophilic matrix; and
wherein the composition exhibits a release profile of the amine-containing compound comprising a substantial portion that is representative of zero-order release kinetics under in vitro conditions.

2. A sustained-release oral pharmaceutical composition comprising within a single dosage form:
a hydrophilic matrix;
a pharmacologically active amine-containing compound;
a pharmaceutically acceptable salt of a non-NSAID cyclic organic acid compound; and
a pharmaceutically acceptable anionic surfactant;
wherein the amine-containing compound, the salt of the cyclic organic acid, and the anionic surfactant are within the hydrophilic matrix.

3. The composition of claim 2 which exhibits a release profile of the amine-containing compound comprising a substantial portion that is representative of zero-order release kinetics under in vitro conditions.

4. The composition of claim 1 wherein the amine group comprises a secondary amine, a tertiary amine, a primary amine, or combination thereof.

5. The composition of claim 4 wherein the amine-containing compound comprises a tertiary amine.

6. The composition of claim 1 wherein the amine-containing compound is an opioid.

7. The composition of claim 6 wherein the opioid is selected from the group consisting of morphine, codeine, hydromorphone, hydrocodone, oxycodone, oxymorphone, desomorphine, diacetylmorphine, buprenorphine, dihydrocodeine, nicomorphine, benzylmorphine, fentanyl, methadone, tramadol, propoxyphene, levorphanol, meperidine, and combinations thereof.

8. The composition of claim 6 wherein the opioid is present in a pain-reducing amount.

9. The composition of claim 1 wherein the amine-containing compound is a non-opioid amine-containing compound.

10. The composition of claim 9 wherein the non-opioid amine-containing compound is selected from the group consisting of dextromethorphan, cyclobenzaprine, benztropine, baclofen, arbaclofen, ritodrine, tizanidine, flurazepam, chlorpheniramine, doxylamine, diphenhydramine, diltiazem, rimantadine, amantadine, memantine, and combinations thereof.

11. The composition of claim 1 wherein the amine-containing compound is a salt comprising a hydrochloride, a bitartrate, an acetate, a naphthylate, a tosylate, a mesylate, a besylate, a succinate, a palmitate, a stearate, an oleate, a pamoate, a laurate, a valerate, a hydrobromide, a sulfate, a methane sulfonate, a tartrate, a citrate, a maleate, or a combination of the foregoing.

12. The composition of claim 1 wherein the salt of the cyclic organic acid is selected from the group consisting of disodium pamoate, sodium saccharin, sodium cyclamate, sodium benzoate, sodium naphthoate, potassium benzoate, and combinations thereof.

13. The composition of claim 2, wherein the pharmaceutically acceptable anionic surfactant is selected from the group consisting of monovalent alkyl carboxylates, acyl lactylates, alkyl ether carboxylates, N-acyl sarcosinates, polyvalent alkyl carbonates, N-acyl glutamates, fatty acid-polypeptide condensates, sulfur-containing surfactants, phosphated ethoxylated alcohols, and combinations thereof.

14. The composition of claim 1 wherein the salt of the cyclic organic acid is present in an amount effective to provide zero-order release kinetics under in vitro conditions.

15. The composition of claim 1 wherein the pharmaceutically acceptable anionic surfactant is present in a release-modifying amount.

16. The composition of claim 1 wherein the single dosage form is a tablet form.

17. The composition of claim 1 wherein the hydrophilic matrix comprises at least one hydrophilic polymeric compound selected from the group consisting of a gum, a cellulose ether, an acrylic resin, a polyvinyl pyrrolidone, a protein-derived compound, and combinations thereof.

18. The composition of claim 2 wherein the amine group comprises a secondary amine, a tertiary amine, a primary amine, or combination thereof.

19. The composition of claim 18 wherein the amine-containing compound comprises a tertiary amine.

20. The composition of claim 2 wherein the amine-containing compound is an opioid.

21. The composition of claim 20 wherein the opioid is selected from the group consisting of morphine, codeine, hydromorphone, hydrocodone, oxycodone, oxymorphone, desomorphine, diacetylmorphine, buprenorphine, dihydrocodeine, nicomorphine, benzylmorphine, fentanyl, methadone, tramadol, propoxyphene, levorphanol, meperidine, and combinations thereof.

22. The composition of claim 20 wherein the opioid is present in a pain-reducing amount.

23. The composition of claim 2 wherein the amine-containing compound is a non-opioid amine-containing compound.

24. The composition of claim 23 wherein the non-opioid amine-containing compound is selected from the group consisting of dextromethorphan, cyclobenzaprine, benztropine, baclofen, arbaclofen, ritodrine, tizanidine, flurazepam, chlorpheniramine, doxylamine, diphenhydramine, diltiazem, rimantadine, amantadine, memantine, and combinations thereof.

25. The composition of claim 2 wherein the amine-containing compound is a salt comprising a hydrochloride, a bitartrate, an acetate, a naphthylate, a tosylate, a mesylate, a besylate, a succinate, a palmitate, a stearate, an oleate, a pamoate, a laurate, a valerate, a hydrobromide, a sulfate, a methane sulfonate, a tartrate, a citrate, a maleate, or a combination of the foregoing.

26. The composition of claim 2 wherein the salt of the cyclic organic acid is selected from the group consisting of disodium pamoate, sodium saccharin, sodium cyclamate, sodium benzoate, sodium naphthoate, potassium benzoate, and combinations thereof.

27. The composition of claim 2 wherein the salt of the cyclic organic acid is present in an amount effective to provide zero-order release kinetics under in vitro conditions.

28. The composition of claim 2 wherein the pharmaceutically acceptable anionic surfactant is present in a release-modifying amount.

29. The composition of claim 2 wherein the single dosage form is a tablet form.

30. The composition of claim 2 wherein the hydrophilic matrix comprises at least one hydrophilic polymeric compound selected from the group consisting of a gum, a cellulose ether, an acrylic resin, a polyvinyl pyrrolidone, a protein-derived compound, and combinations thereof.
